(19) United States
(12) Patent Application Publication
(10) Pub. No.: US 2004/0115655 A1
(43) Pub. Date: Jun. 17, 2004

(54) Method for analyzing the nucleotide sequence of nucleic acids

(76) Inventor: Diane D. Ilsley, San Jose, CA (US)

Correspondence Address: AGILENT TECHNOLOGIES, INC., Legal Department, M/S DL429, Intellectual Property Administration, P.O. Box 7599, Loveland, CO, 80537-0599, US

(21) Appl. No.: 10/320,197

(22) Filed: Dec. 16, 2002

(51) Int. Cl. C12Q 1/68; C12P 19/34

(52) U.S. Cl. 435/6; 435/91.2

(57) ABSTRACT

Provided are methods for deciphering the sequence of a nucleic acid by synthesizing fragments with pol α-primase and analyzing the fragments. The fragments are preferably short Xmers that are analyzed by mass spectrometry.

### FIELD OF THE INVENTION

The present invention relates to the field of nucleic acid sequencing, particularly through the use of mass spectrometry.

### BACKGROUND OF THE INVENTION

A number of enzymatic and chemical techniques exist to determine the nucleotide sequence of nucleic acids and to identify sequence mutations. Some of these include direct sequencing, single-stranded conformation polymorphism, ligase and polymerase extension assays, DNA microarrays, and mass spectrometry using cleavable mass tags.

Some of these methods synthesize fragments complimentary to the target nucleic acid and then ascertain the sequence of the target by analyzing the fragments. One such method is the pioneering method of F. Sanger et al. (Proc. Natl. Acad. Sci. USA 74:5463-67, 1977), also called the dideoxy method. The Sanger method uses a labeled DNA primer, and runs four separate reaction mixtures each containing the dideoxy nucleotide of one of the dNTPs. The resulting DNA is then denatured, separated by electrophoresis and analyzed to ascertain the sequence of the target.

The general idea behind the Sanger method can be used in a variety of ways to ascertain the sequence of nucleic acids. For example, the fragments obtained via the Sanger method can be analyzed by mass spectrometry. See e.g. U.S. Pat. No. 6,238,871 (hereinafter, “Koster”) (incorporated herein by reference). In one method for example, a mass spectrometer is used to analyze nested fragments obtained by base-specific chain termination.

A general problem with the current sequencing methods, including those that utilize the dideoxy method of Sanger, is that they require some prior knowledge of the target nucleic acid sequence. For example, in one method, short primers can be annealed to a target nucleic acid, which are then extended by a polymerase. The extension is then terminated by incorporation of a dideoxy nucleotide, followed by analysis of the fragments to ascertain the sequence of the target. To synthesize the short fragments, however, primers are needed. Since the sequence of the target is not known, a set of 4,096 6-mers representing every possible sequence must be synthesized, increasing the cost of the process.

Another problem with priming is that the reaction is performed at 37° C., which is above the melting temperature (Tm) for annealing of the primers, such as heptamers, to the DNA template. In addition, a strong sequence influence exists (GC-rich regions are primed at a higher frequency than AT-rich regions). In areas of the template where there is secondary structure, the primer may not anneal. Hence, secondary structure can have a strong influence as to where synthesis is initiated and terminated, which can result in an incomplete synthesis of the second strand. Furthermore, random priming requires the synthesis of the random primers on a DNA synthesizer, followed by exogenous annealing, which can be burdensome.

In another approach, the target sequence has to be digested by a restriction enzyme and inserted into a vector, followed by primer attachment based on the sequence of the vector. See e.g. Koster. The problem with this approach, in addition to its many steps, is that fragments, particularly short Xmers, cannot be synthesized randomly all along the target since the sequence of the target DNA is unknown.

There is a need for methods and reagents that can be applied generically without any prior sequence knowledge to determine the sequence of a target nucleic acid and the presence, location, or identity of mutations in a target nucleic acid.

### SUMMARY OF THE INVENTION

In one aspect, the present invention provides a method for analyzing sequence of a target nucleic acid comprising the steps of:

a) synthesizing a fragment on the target nucleic acid with pol α-primase, wherein the fragment includes an RNA primer synthesized by pol α-primase;

b) separating the fragment from the target nucleic acid; and

c) analyzing the fragment to ascertain the sequence of the target nucleic acid. Preferably, the synthesizing step involves synthesis of fragments at random locations on the target nucleic acid; the fragments are substantially from about 6 to about 12 nucleotides in length; and analysis is carried out by a mass spectrometer.

In another aspect, the present invention provides a method for analyzing a sequence of a target DNA comprising the steps of:

a) synthesizing complimentary fragments on the target DNA at with pol α-primase in a reaction mixture containing NTPs and at least one of four ddNTPs, in the optional presence of dNTPs;

b) separating the fragments from the target DNA;

c) measuring the mass of the fragments; and

d) ascertaining the sequence of the fragments from the mass.

In another aspect, the present invention provides a method for preparing fragments for sequence analysis by mass spectrometry comprising the step of synthesizing with pol α-primase fragments that are substantially from about 6 to about 12 nucleotides at random locations on a target nucleic acid.

### DESCRIPTION OF THE FIGURE

FIG. 1 illustrates synthesis of RNA primers all along the target DNA, followed by addition of a single ddNTP, by using pol α-primase.

### DETAILED DESCRIPTION OF THE INVENTION

The present invention provides a method for the analysis of a nucleic acid sequence, such as a high resolution mass spectrometry technique, by synthesizing fragments without the use of a primer. The method of the present invention does not need to rely on short oligonucleotide primers or target sequence information because priming is done enzymatically and randomly, using pol α-primase. Pol α-primase is disclosed in the art. See e.g. U.S. Pat. No. 6,096,499 (hereinafter, “Kozlowski”), incorporated herein by reference.

Rather than ascertaining the sequence of a target nucleic acid for priming or synthesizing every possible combination of primers, pol α-primase can be used to synthesize short fragments randomly. A fragment refers to any complimentary nucleic acid, shorter than the full length of the target nucleic acid, or of the same length, that is of a size suitable for the proper analysis procedure. For example, when using mass spectrometry for analysis, short Xmers are preferred because mass spectrometry is particularly effective at volatilizing small molecules. Fragment can also include nucleic acid that is substantially of the same length as that of the target nucleic acid, which for example can then be subsequently digested by an endonuclease to obtain shorter fragments.

Pol α-primase is a eukaryotic DNA polymerase capable of binding single-stranded DNA and initiating DNA synthesis de novo. Pol α-primase is a four-subunit complex (49, 58, 68 and 180 KD) that contains two enzymatic activities: DNA primase and polymerase. The larger subunits, 180 and 68, are responsible for polymerase activity while the smaller subunits, 49 and 58, are responsible for the primase activity.

The subunits of DNA primase have been cloned as cDNA from both mice and humans, and their sequences are reported in EMBL/GenBank computer-accessible public databases, among others. See Kozlowski. Additionally, the synthesis of oligonucleotides by the subunits of pol α-primase have been studied in the art. For example, one study measures the inhibition of the polymerase activity of pol α-primase with acyclic nucleotide analogs, such as acyclovir, canciclovir and penciclovir. See Ilsely et al., Biochemistry 1995, 34, 2504-10. Kozlowski discloses methods for identifying modulators and modifiers of the primase unit of pol α-primase.

Without being bound by any theory, pol α-primase is thought to synthesize the lagging strand during DNA replication in vivo. See Lodish et al., Molecular Cell Biology, 3^{rd} ed., p. 379. The primase activity synthesizes short oligonucleotides, about 7-10 nucleotides in length, during initiation of DNA replication and elongation of the lagging strand. DNA polymerase a then elongates the RNA primer to complete the synthesis of Okazaki fragments. See Stillman, B., Ann. Rev. Cell Biol. 5:197 (1989). Okazaki fragments are then extended by pol 6-primase and pol α-primase, allowing DNA pol α-primase to recycle and initiate another Okazaki fragment on the lagging strand. See Waga, S. and Stillman, B., Nature 369:207 (1994). A unique property of DNA primase is the ability to synthesize oligonucleotides de novo on a template by the formation of an initial dinucleotide. DNA primase initiates synthesis with a triphosphate moiety at the 5′ end. See Gronostajski et al., J. Biol. Chem. 259: 9479 (1984).

According to the current model, pot α-primase first binds the DNA template, and then slides along the single-stranded DNA a short distance until it has bound two NTPs. At this point, the enzyme becomes immobilized on the template and becomes poised to begin synthesis of an RNA primer. Unlike prokaryotic DNA primases that show preferences for specific sequences for initiation, eukaryotic primases initiate synthesis at many different sequences. See Kirk et al., Biochemistry, 36:6725-6731, 1997.

Initiation by pot α-primase is sequence independent, and exogenous primers are not necessary. (See Id.). As long as the concentration of NTPs is similar to those found in vivo, the enzyme will initiate randomly all along the template as supposed to at a specific sequence. (At low concentrations, the enzyme prefers pyrimidine rich regions.). The concentration of NTPs used is preferably in the 1 millimolar range. (See Id.).

On single-stranded DNA, primase synthesizes short RNA oligomers of approximately 7-10 nucleotides in length. Once a primer is synthesized, it is transferred intramolecularly to the polymerase active site.

During the transfer of the primer to the polymerase site, the enzyme complex remains bound to the primer-template. The lack of dissociation from the primer-template renders the primer annealed to the template despite its short length. The association of the enzyme to the primer-template makes the Tm of the primer-template less relevant. The primer-template hybrid, which has a relatively low Tm and dissociates rapidly, becomes stable at higher temperatures and lower ion concentrations. The dissociation of the enzyme from the hybrid becomes a new rate-limiting step.

The secondary structure of the template is reduced as the enzyme slides along the single-stranded template. The reduction in secondary structure may further increase the stability of the primer-template complex.

One of skill in the art would appreciate that pot α-primase obtained from many different eukaryotic organisms may be used in the present invention. Preferably, pol α-primase from calf thymus is used. See Thompson et al., Biochemistry 34, 11198-11203 (1995). The synthesis of the second strand with pol α-primase can be carried out by incubating a buffered reaction mixture containing single stranded cDNA, pol α-primase, NTPs and dNTPs. The reaction is preferably carried out at a temperature of about 37° C., with a buffer, such as Tris, containing various ions, at a pH of about 7.5. EDTA or formamide can be used to stop the reaction when desired. See Ilsely et al., Biochemistry, 34, 2504-10, 1995.

The processivity of pol α-primase, at about 50-200 nucleotides, is low. When longer DNA fragments are desired, a second DNA polymerase may be added to further extend the primer. Depending on the length of the first DNA fragment and the time when the second DNA polymerase is added, the DNA fragment may be generated by using pol α-primase, a subsequently added DNA polymerase or a combination thereof. In either case, the second DNA polymerase uses the primer synthesized by pol α-primase, even if pol α-primase does not extend the primer.

Preferably, the second DNA polymerase is added after the extension of the primer by pol α-primase. The intramolecular transfer of the primer to the polymerase unit of the pol α-primase without dissociation allows for the primer to be annealed at all times. In one embodiment, pol α-primase synthesizes the primer and extends the primer from about 30 to about 70 nucleotides, followed by the addition of a second DNA polymerase to complete the synthesis of the fragment.

One of skill in the art would appreciate that many DNA polymerases can be used as the second DNA polymerase. Depending on the length of the strand, DNA polymerases with different processivity can be used. Processivity refers to the ability of a DNA polymerase to incorporate nucleotides without dissociating from the template. See U.S. Pat. No. 5,972,603 (hereinafter, “Bedford”). Klenow fragments can be used which has an average processivity of about 250 nucleotides. See Johnson, K., Annu. Rev. Biochem 62:685-713. See Bedford. A T4 or Taq DNA polymerase can also be used.

The processivity of the DNA polymerase used can also be altered. U.S. Pat. No. 5,728,526 (hereinafter, “George, Jr.”) discloses that a decrease in salt concentration or an increase in nucleotide concentrations increase processivity.

In one embodiment, the DNA polymerases used has displacement activity. Displacement activity is the ability to displace and remove oligonucleotides annealed to the template. See George, Jr. As synthesis of the DNA fragment approaches the 3′ end, the downstream fragments are displaced. The present invention prefers the use of Klenow fragment over other polymerases with displacement activity, such as Bst, Vent and MMLV. In one embodiment, Klenow fragment lacking exonuclease activity is used since exonuclease activity can result in the digestion of the ends of the strands and an undesirably shortened cDNA. The lack of exonuclease activity in Klenow fragment eliminates the need to protect the cDNA strand by capping.

In another embodiment, the second DNA polymerase added does not have displacement activity, resulting in non-overlapping set of fragments. If the reaction is continued to completion, then fragments can be synthesized that theoretically cover the entire target DNA. These fragments can then be used to ascertain the sequence of the target.

Pol α-primase can be used with various analysis techniques to ascertain the sequence of the target. These techniques include, inter alia, use of mass tags, fluorescent dyes, radioactive labels and NMR tags. Preferably, the chain termination (dideoxy method) of Sanger is used in the present invention. Some of the methods for sequencing nucleic acids disclosed in Koster (incorporated herein by reference) may be used with the present invention, as one of skill in the art would appreciate.

The fragments obtained can be overlapping, semi-overlapping or non-overlapping, as disclosed in U.S. Pat. No. 6,218,118 (hereinafter, “Sampson”), incorporated herein by reference. To obtain overlapping fragments, the reaction can be repeated or carried out simultaneously in different chambers. Since pol α-primase initiates synthesis randomly, the fragments would most likely be different each time, allowing for overlap. Furthermore, the conditions can be manipulated to make pol α-primase prefer a region over another, as described above. See Kirk et al., Biochemistry, 36:6725-6731, 1997. To obtain non-overlapping primers, the synthesis reaction can be carried out to its completion.

The use of pol α-primase allows for other manipulations of the analysis process, further facilitating sequencing. Depending on the sequencing technique used, the desired fragments may be short Xmers, substantially from about 6 to about 12 nucleotides, more preferably from about 8 to about 11 nucleotides in length. In a preferred embodiment, the method of the present invention synthesizes short Xmers followed by their mass analysis. Longer fragments can be used with automated sequencers that use fluorescent dyes.

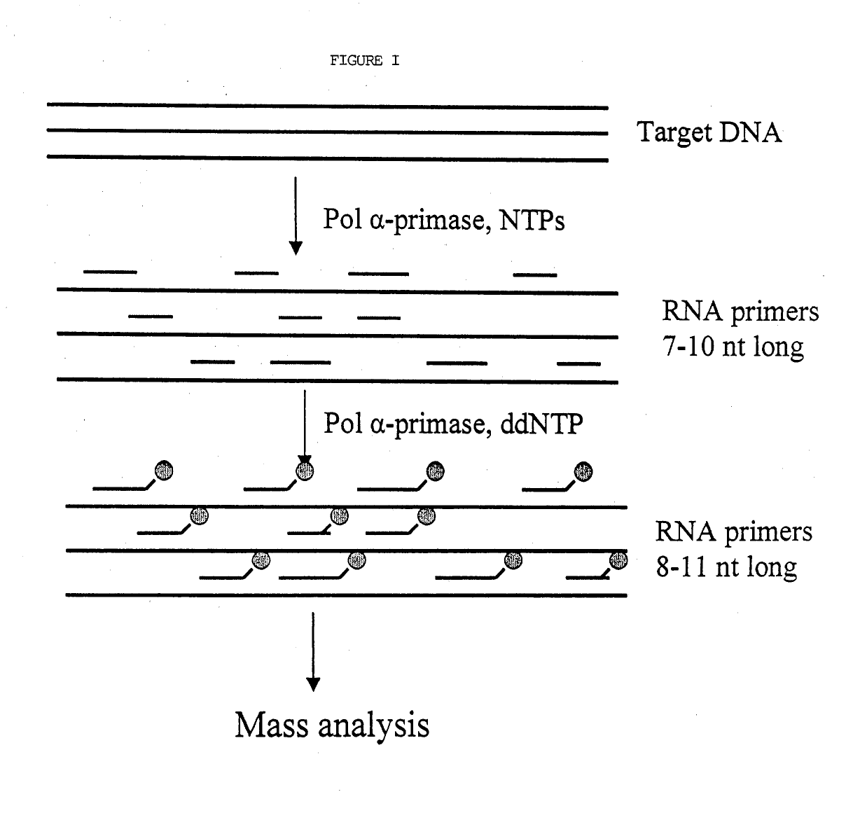

The synthesis of the fragments with pol α-primase can be carried out to obtain fragments of desired lengths. In one embodiment, a second DNA polymerase such as Klenow fragment is not added to avoid significantly long fragments, while in another embodiment the ratio of dideoxy nucleotides is increased in relation to dNTPs to result in shorter fragments. In a preferred embodiment, only NTPs and ddNTPS are used, allowing for the synthesis of short RNA fragments which are terminated by addition of a single ddNTP (FIG. 1).

Another manner of obtaining short fragments is to use pol α-primase with Klenow fragment to obtain a long fragment, followed by digestion of the long fragment to obtain short fragments. A restriction enzyme (endonuclease) can then be used to cleave the fragment into shorter fragments. An exonuclease can also be used to obtain shorter fragments. The long fragment can also be treated with chemical agents to obtain shorter fragments. The resulting non-overlapping fragments can then be analyzed.

Sampson, incorporated herein by reference, discloses and teaches use of mass tags to analyze the sequence of nucleic acids with the dideoxy method. The method of Sampson generally uses hexamer primers and extends the hexamers by a single mass tagged nucleotide. The mass of the resulting heptamers is then analyzed in a mass spectrometer and compared to the hexamer precursors to determine the sequence of the nucleic acid.

Mass tags can be used in a similar fashion in the present invention to determine the sequence of the nucleic acid, by synthesizing fragments, and then ascertaining the sequence of the target by analyzing the mass of the fragments. See generally P. F. Crain, “Mass Spectrometric Techniques in Nucleic Acid Research, ” Mass Spectrometry Review 9, 505-554 (1990) (incorporated herein by reference). The reaction can be run in a single chamber, or be split up into at least four chambers with each chamber having a single dideoxynucleotide. In one embodiment, when using mass tags, the level of the dideoxynucleotides and the length of the reaction is such to give Xmers substantially in the range of about 6 to about 12, more preferably of about 8 to about 11 nucleotides. One of skill in the art would appreciate that a minor portion of the fragments can in some instances be outside of this range.

A problem with mass spectrometry is the ambiguity of the process, since the mass of the fragment does not reveal the location of the nucleotides. Ambiguity that arises from using mass spectrometry can be decreased by the method of the present invention. The ambiguity can be decreased by tagging the NTPs, dNTPs and/or ddNTPs used during synthesis. Ambiguity can also be decreased by using a different mass tag for NTPs, dNTPs and ddNTPs, wherein the distinct mass of the ddNTP confirms the position of at least one nucleotide.

In a simplified illustration, short fragments are synthesized using pol a-primase, NTPs and tagged ddNTPS. The reaction is conducted in four different chambers, each containing one tagged ddNTP. The resulting fragments are then separated from the target sequence and put into a mass spectrometer. The masses of the fragments are then analyzed to ascertain the sequence of the target.

The fragments can be separated from the nucleic acid by methods well known in the art. One approach for separating the fragments is denaturation. The double stranded segments of the nucleic acid can be denatured by raising the temperature, lowering the ion concentration, or using an agent that disrupts hydrogen bonds, such as urea and formamide. The fragments can also be separated enzymatically, such as by using a helicase.

For optimal results, the fragments are purified by crystallization out of a suitable solvent, such as ethanol. The fragments can also be purified by using electrophoresis, liquid chromatography or high speed gel filtration. Before mass spectrometry analysis, it may be desirable to condition the fragments, such as to decrease the amount of energy required for ionization. See Sampson.

Analysis of fragments of nucleic acid by mass spectrometry to ascertain the sequence of a nucleic acid is well known in the art. See e.g. Sampson and Koster. The fragments, preferably short Xmers, are put in a mass spectrometer. Some masses will precisely give the sequence of the fragment, while others will be ambiguous and give a mass for several different sequences. By using a tagged dideoxy nucleotide, such ambiguity can be reduced. Additionally, the process can result in overlapping fragments, thus eventually revealing the sequence of the target. Data of known fragments can be gathered and compared to that of the target fragment. Mass spectrometry techniques known in the art, such as fast bombardment technique (FAB), plasma desorption (PD), electrospray/ionspary (ES) and matrix-assisted laser desorption/ionization can be used (MALDI). See e.g. Koster and Sampson.

One of skill in the art can create an algorithm to determine the sequence of the target nucleic acid by the present invention. An algorithm can be made by any method well known in the art. In one embodiment, masses of all known fragments are pre-recorded in a database and compared to the mass of synthesized fragments. If a dideoxy nucleotide is used, the position of at least one nucleotide is known. To decipher the exact sequence of the target nucleic acid, additional fragments can be synthesized, with the data generated from each run being analyzed by a software that uses an algorithm known in the art. Comparisons of the data overtime will eventually give the exact sequence of the target nucleic acid.

One of skill in the art would appreciate that the method of the present invention does not require use of a tag. Rather, the tag is used to decrease the ambiguity of the fragments and facilitate sequence analysis.

In addition to a mass tag, a fluorescent dye can also be used for sequencing. Each NTP, dNTP and/or ddNTP can be dyed with a different color, allowing for sequencing based on the color of the dye. An advantage of fluorescent dyes over mass tags is a decrease in ambiguity, in that the color of the nucleotide also identifies the position of the nucleotide. Fluorescent dyes, however, have the disadvantage of requiring more preparation, as they usually have to be run through a gel.

In one embodiment, in which fluorescent dyes are used, the RNA, DNA or a hybrid labeled with four different dyes is loaded in vertical slabs of acrylamide. Multiple lanes can be run on a single gel. The dyed nucleic acid then migrates through the gel and separates according to size. The dyed nucleic acid passes through the lower region of the gel where a laser beam continuously scans across the gel. The laser results in excitation of the dye and the emission of a particular wavelength. The resulting signal is then converted to a digital signal, by techniques well known in the art. An automated DNA sequencer, such as ABI 373 DNA or ABI prism 377 DNA sequencer (Applied Biosystems Inc., Foster City, Calif.), can be used. Other automated nucleic acid sequencers can also be used.

The fragments used with this method are usually longer than those analyzed by mass spectrometry in part because of a decrease in ambiguity. The fragments are preferably of such a length long enough to allow for efficient sequencing, yet short enough to run through a gel and provide a readable signal. Preferably, the length of such fragment is about 15 to about 60 nucleotides. The preference in length may vary if the method is modified.

In another embodiment, radioactive labeled tags are used. Examples of such tags include ³²P and ³³p . In one embodiment, the resulting fragments are run though gel, followed by exposure to X-ray film. The banding pattern can then be visualized, usually through manual autoradiography.

The present invention also encompasses methods that do not use the dideoxy method. For example, the general technique of Maxam and Gilbert can be used to analyze the sequence of the nucleic acid. See Methods of Enzymology 65, 499-560 (1980). In that technique, a 5′ end-labeled DNA is prepared for sequencing and subjected to four different chemical reactions that break up the DNA. The end-labeled DNA fragment of the Maxam and Gilbert method can be prepared by using the pol α-primase of the present invention. Pol α-primase can be used with or without Klenow fragment to obtain long fragments, followed by labeling the 5′ end and denaturation to obtain a single strand. The resulting labeled single strand is then analyzed by the Maxam-Gilbert technique. The Maxam-Gilbert technique can be generalized to apply to methods known in the art that sequence through degradation of a labeled nucleic acid. As discussed, such a labeled nucleic acid can be prepared by using pol α-primase.

In the presence of NTPs and dNTPs, Pol α-primase in reality synthesizes a DNA fragment that has an RNA portion, since the RNA portion acts as a primer. In one embodiment, in subsequent analysis, the RNA portion can be removed, for example, by using an enzyme that digests RNA, such as RNAse H. The DNA fragment may also be analyzed with the RNA portion.

In a preferred embodiment, illustrated in FIG. 1, an RNA fragment is synthesized on the target nucleic acid with primase unit of Pol α-primase, and allowed to extend by one ddNTP. The resulting RNA primer plus one ddNTP is then analyzed in a mass spectrometer. The use of RNA fragments for analysis has advantages since the primase unit of Pol α-primase lacks specificity in making the RNA fragment. Pol α-primase readily incorporates modified nucleotide analogs onto a primase-synthesized primer and is not that selective in incorporating the first dNTP or ddNTP. See Ilsely et al., Biochemistry 1995, 34, 2504-10. For example, Pol α-primase polymerizes dGTP 100-fold more readily than it polymerizes acyclovir triphosphate when an exogenously added DNA primer-template is used as a substrate. In contrast, Pol α-primase prefers to polymerize dGTP rather than acyclovir triphosphate by a factor of 5 during polymerization of the first nucleotides onto the primase-synthesized primer. A second example, the incorporation of araCTP by Pol a onto an exogenously added DNA primer-template, results in chain termination. In contrast, araCTP is incorporated into internucleotide linkages during elongation of primase-synthesized primers.

This lack of specificity is advantageous because polymerization of a single dNTP that contains a mass tag or other modification can be utilized to alter the mass of each RNA primer in order to decrease the number of mass coincidences, i.e., ambiguity. Furthermore, this lack of specificity allows for the integration of the base at the 3′ end of the RNA primer. Because Pol α exhibits altered specificity during primase-coupled Pol α activity, modified chain-terminating ddNTPS can easily be incorporated.

The present invention also provides for kits. Kits are prepared containing all the necessary ingredients for the synthesis of fragments with pol α-primase. Each kit preferably contains at least NTPs and ddNTPs, with at least some of the bases being tagged.

Having thus described the invention with reference to particular preferred embodiments, those in the art can appreciate modifications to the invention as described and illustrated that do not depart from the spirit and scope of the invention as disclosed in the specification. The embodiments are set forth to aid in understanding the invention but are not intended to, and should not be construed to, limit its scope in any way. The embodiments do not include detailed descriptions of conventional methods. Such methods are well known to those of ordinary skill in the art and are described in numerous publications. Detailed descriptions of conventional methods relating to manipulations of DNA, RNA, and proteins can be obtained from numerous publications, including Sambrook, J. et al., Molecular Cloning: A Laboratory Manual, 2^{nd} ed., Cold Spring Harbor Laboratory Press (1989). Koster and Sampson are incorporated herein by reference regarding their disclosures on use of mass spectrometry to analyze sequences and their disclosure on general sequencing techniques. Additional disclosure on mass spectrometry is provided in U.S. Pat. No. 5,872,659 (“Patterson”) and U.S. Pat. No. 5,064,754 (“Mills”), also incorporated by reference. Additionally, all references mentioned herein are incorporated by reference in their entirety, particularly in regard to pertinent subject matter referred to when discussing or citing that reference.

What is claimed is:

1. A method for analyzing sequence of a target nucleic acid comprising the steps of:
a) synthesizing a fragment on the target nucleic acid with pol α-primase, wherein the fragment includes an RNA primer synthesized by pol α-primase;
b) separating the fragment from the target nucleic acid; and
c) analyzing the fragment to ascertain the sequence of the target nucleic acid.

2. The method of claim 1, wherein the synthesizing step involves synthesis of fragments at random locations on the target nucleic acid.

3. The method of claim 2, wherein the fragments are substantially from about 6 to about 12 nucleotides in length.

4. The method of claim 3, wherein the fragments are substantially from about 8 to about 11 nucleotides in length.

5. The method of claim 3, wherein the synthesizing a fragment step is terminated by using a ddNTP.

6. The method of claim 5, wherein the fragment is an RNA primer that is terminated by a single ddNTP in the absence of dNTPs.

7. The method of claim 3, wherein the analyzing step is carried out by a mass spectrometer.

8. The method of claim 1, further comprising a step of removing the RNA portion of the fragment before the analyzing step.

9. A method for analyzing a sequence of a target DNA comprising the steps of:
a) synthesizing complimentary fragments on the target DNA with pol α-primase in a reaction mixture containing NTPs and at least one of four ddNTPs, in the optional presence of dNTPs;
b) separating the fragments from the target DNA;
c) measuring the mass of the fragments; and
d) ascertaining the sequence of the fragments from the mass.

10. The method of claim 9, wherein the ddNTP is mass tagged.

11. The method of claim 9, wherein the fragments are substantially from about 6 to about 12 nucleotides in length.

12. The method of claim 11, wherein the fragments are substantially from about 8 to about 11 nucleotides in length.

13. The method of claim 9, wherein the fragment are RNA fragments having a single ddNTP at 3′ end.

14. A method for preparing fragments for sequence analysis by mass spectrometry comprising the step of synthesizing with pol α-primase fragments that are substantially from about 6 to about 12 nucleotides long at random locations on a target nucleic acid.

15. The method of claim 14, wherein the fragments are substantially from about to about 11 nucleotides in length.

16. The method of claim 14, wherein the fragments are RNA fragments having a single ddNTP at 3′ end.
